(19) United States
(12) Patent Application Publication
(10) Pub. No.: US 2026/0109691 A1
(43) Pub. Date: Apr. 23, 2026

(54) PROCESS FOR MANUFACTURING ALOGABAT

(71) Applicant: Hoffmann-La Roche Inc., Little Falls, NJ (US)

(72) Inventors: Maud GUILLEMOT-PLASS, Village-Neuf (FR); Christian JENNY, Basel (CH); Matthias KOERNER, Grenzach-Whylen (DE); Isabelle Georgette Huguette MODOLO-CHELLAT, Riehen (CH); Michelangelo SCALONE, Birsfelden (CH)

(73) Assignee: Hoffmann-La Roche Inc., Little Falls, NJ (US)

(21) Appl. No.: 19/123,664

(22) PCT Filed: Oct. 23, 2023
(86) PCT No.: PCT/EP2023/079404

§ 371 Date: Apr. 23, 2025

(30) Foreign Application Priority Data
Oct. 25, 2022 (EP) 22203478.7

(51) Int. Cl. C07D 413/14; B01J 31/24; C07D 405/12

(52) U.S. Cl. CPC C07D 413/14; B01J 31/2409; C07D 405/12; B01J 2231/4205; B01J 2231/4288; B01J 2531/0241; B01J 2531/824

(57) ABSTRACT

The invention relates to novel processes for manufacturing 6-[[5-methyl-3-(6-methyl-3-pyridyl)isoxazol-4-yl]methoxy]-N-tetrahydropyran-4-yl-pyridazine-3-carboxamide (1), or a pharmaceutically acceptable salt thereof. The processes according to the invention are particularly suitable for large-scale manufacturing of the compound of formula 1 under GMP conditions.

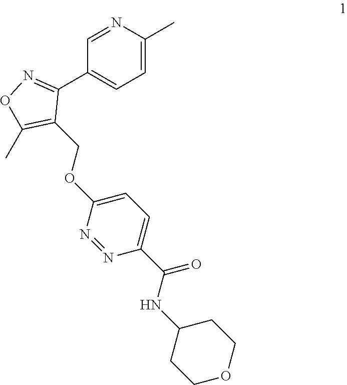 [SMILES: Cc1ccc(-c2noc(C)c2COc2ccc(C(=O)NC3CCOCC3)nn2)cn1]

### FIELD OF THE INVENTION

The invention relates to novel processes for manufacturing 6-[[5-methyl-3-(6-methyl-3-pyridyl)isoxazol-4-yl]methoxy]-N-tetrahydropyran-4-yl-pyridazine-3-carboxamide (1), or a pharmaceutically acceptable salt thereof.

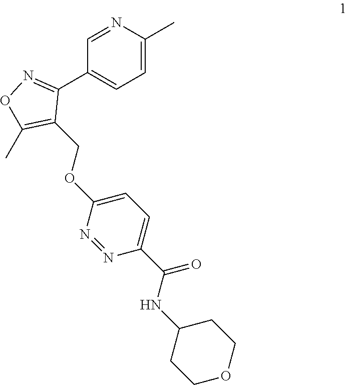 [SMILES: Cc1ccc(-c2noc(C)c2COc2ccc(C(=O)NC3CCOCC3)nn2)cn1]

The process according to the invention is particularly suitable for large-scale manufacturing of the compound of formula 1 under GMP conditions.

### BACKGROUND OF THE INVENTION

6-[[5-methyl-3-(6-methyl-3-pyridyl)isoxazol-4-yl]methoxy]-N-tetrahydropyran-4-yl-pyridazine-3-carboxamide (1) is a positive allosteric modulator (PAM) of the GABA_{A} α5 receptor (WO2018104419), which is currently being investigated in clinical trials for the treatment of Autism Spectrum Disorder (ASD).

Compound 1 is also known under the INN alogabat (WHO Drug Information, Vol. 35, No. 2, 2021, 366).

WO2018104419 discloses a laboratory scale (6 mg) process for manufacting alogabat. It has been found that this process is not suitable for industrial-scale manufacturing of alogabat, i.a. due to use of toxic solvents, such as DMF, the need for silica gel chromatography, formation of side products, and low yields.

Accordingly, there is a high unmet need for new processes for manufacturing alogabat, in order to be able to provide patients with this new treatment option for ASD.

### SUMMARY OF THE INVENTION

The present invention provides improved processes for manufacturing alogabat (1), which overcome the problems outlined above.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims and the abstract), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims and the abstract), or to any novel one, or any novel combination, of the steps of any method or process so disclosed.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

### Manufacturing Processes

### Process A

In a first aspect, the present invention provides a process for manufacturing alogabat (1), or a pharmaceutically acceptable salt thereof,

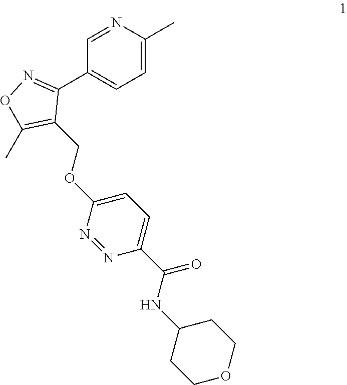 [SMILES: Cc1ccc(-c2noc(C)c2COc2ccc(C(=O)NC3CCOCC3)nn2)cn1]

comprising reacting [5-methyl-3-(6-methyl-3-pyridyl)isoxazol-4-yl]methanol (2)

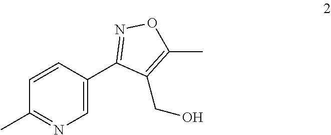

with 6-chloro-N-tetrahydropyran-4-yl-pyridazine-3-carboxamide (3)

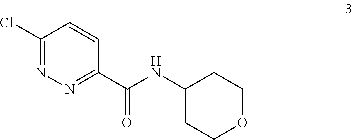 [SMILES: O=C(NC1CCOCC1)c1ccc(Cl)nn1]

in the presence of a base, to afford said alogabat (1).

In one embodiment, said base used in the reaction of compound 2 with compound 3 is selected from Cs₂CO₃, NaO^{t}Bu, and sodium hydride.

In a preferred embodiment, said base is selected from NaO^{t}Bu and sodium hydride.

In a particularly preferred embodiment, said base is sodium hydride.

In one embodiment, the reaction of compound 2 with compound 3 is conducted in an aprotic organic solvent.

In one embodiment, said aprotic organic solvent is selected from N,N-dimethylacetamide, THF, and 2-methyltetrahydrofuran.

In a preferred embodiment, said aprotic organic solvent is selected from THF and 2-methyltetrahydrofuran.

In a particularly preferred embodiment, said aprotic organic solvent is 2-methyltetrahydrofuran.

The process for manufacturing alogabat described in WO2018104419 requires purification of the final product by silica gel chromatography. However, silica gel chromatography is not suitable for industrial scale manufacturing of pharmaceutical compounds. Alogabat as obtained by the process of the present invention has an impurity profile that allows it to be purified to pharmaceutical grade quality under GMP conditions by filtration and crystallization.

Thus, in one embodiment of the process according to the invention, alogabat (1) is purified by filtering the reaction mixture over activated charcoal.

In one embodiment of the process according to the invention, alogabat (1) is purified by crystallization.

In a preferred embodiment, said crystallization is crystallization from 1-propanol.

In one embodiment of the process according to the invention, alogabat (1) is purified by (i) filtering the reaction mixture over activated charcoal, followed by (ii) crystallization.

In one embodiment of the process according to the invention, the starting material 6-chloro-N-tetrahydropyran-4-yl-pyridazine-3-carboxamide (3) is obtained by reacting 3,6-dichloropyridazine (4)

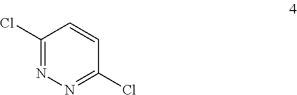

with tetrahydro-2H-pyran-4-amine (5)

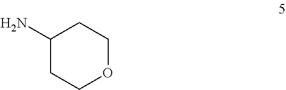

and carbon monoxide in the presence of a base and a palladium catalyst.

In one embodiment, said palladium catalyst used in the reaction of compound 4 with compound 5 and carbon monoxide is selected from PdCl₂(dppp), PdCl₂(P(p-FC₆H₄)₃)₂, PdCl₂(xantphos), PdCl₂(PPh₃)₂, PdCl₂(dppf), and Pd(amphos)Cl₂.

In a preferred embodiment, said palladium catalyst is PdCl₂(dppp).

In one embodiment, said base used in the reaction of compound 4 with compound 5 and carbon monoxide is selected from triethylamine and N-ethyldiisopropylamine.

In a preferred embodiment, said base used in the reaction of compound 4 with compound 5 and carbon monoxide is N-ethyldiisopropylamine.

In one embodiment, the reaction of compound 4 with compound 5 and carbon monoxide is conducted in a solvent selected from THF, 2-propanol, ethyl acetate, toluene, and ethanol.

In a preferred embodiment, said solvent is selected from THF and 2-propanol.

In a particularly preferred embodiment, said solvent is 2-propanol.

In one embodiment, the process according to the invention is as depicted in Scheme 1.

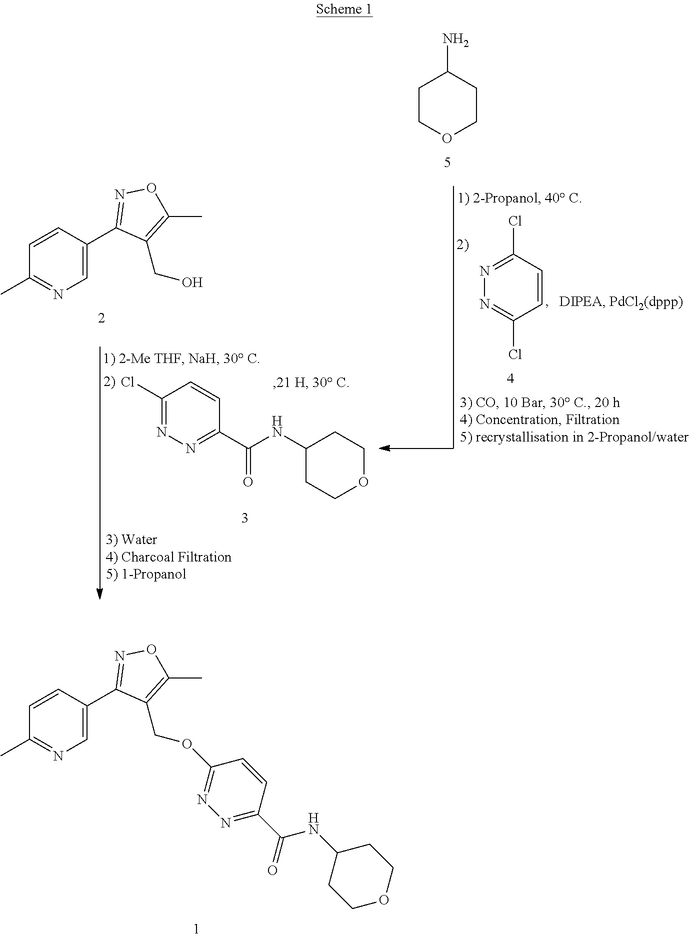

### Process B

6-chloro-N-tetrahydropyran-4-yl-pyridazine-3-carboxamide (3) is a crucial starting material in the manufacturing process of alogabat (1) according to the invention. It is also an aspect of the present invention to provide a process that allows an industrial scale manufacturing of compound 3.

Thus, in one aspect, the present invention provides a process for manufacturing 6-chloro-N-tetrahydropyran-4-yl-pyridazine-3-carboxamide (3),

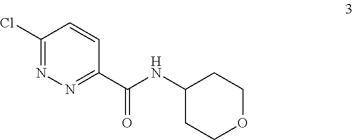 [SMILES: O=C(NC1CCOCC1)c1ccc(Cl)nn1]

comprising reacting 3,6-dichloropyridazine (4)

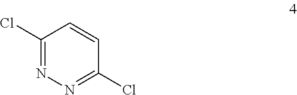

with tetrahydro-2H-pyran-4-amine (5)

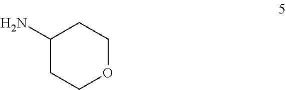

and carbon monoxide in the presence of a base and a palladium catalyst to afford said 6-chloro-N-tetrahydropyran-4-yl-pyridazine-3-carboxamide (3).

In one embodiment, said palladium catalyst used in the reaction of compound 4 with compound 5 and carbon monoxide is selected from PdCl₂(dppp), PdCl₂(P(p-FC₆H₄)₃)₂, PdCl₂(xantphos), PdCl₂(PPh₃)₂, PdCl₂(dppf), and Pd(amphos)Cl₂.

In a preferred embodiment, said palladium catalyst used in the reaction of compound 4 with compound 5 and carbon monoxide is PdCl₂(dppp).

In one embodiment, said base used in the reaction of compound 4 with compound 5 and carbon monoxide is selected from triethylamine and N-ethyldiisopropylamine.

In a preferred embodiment, said base used in the reaction of compound 4 with compound 5 and carbon monoxide is N-ethyldiisopropylamine.

In one embodiment, the reaction of compound 4 with compound 5 and carbon monoxide is conducted in a solvent selected from THF, 2-propanol, ethyl acetate, toluene, and ethanol.

In a preferred embodiment, said solvent is selected from THF and 2-propanol.

In a particularly preferred embodiment, said solvent is 2-propanol.

In one aspect, the present invention provides using the process for manufacturing compound 3 in the manufacture of alogabat (1).

### Process C

In one aspect, the present invention provides a process for manufacturing alogabat (1), or a pharmaceutically acceptable salt thereof,

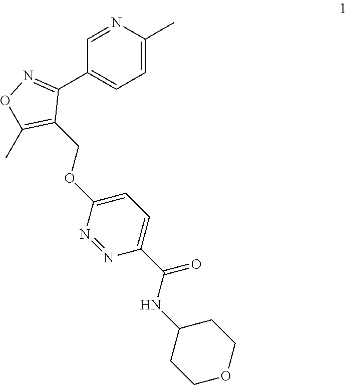 [SMILES: Cc1ccc(-c2noc(C)c2COc2ccc(C(=O)NC3CCOCC3)nn2)cn1]

comprising reacting 4-[(6-chloropyridazin-3-yl)oxymethyl]-5-methyl-3-(6-methyl-3-pyridyl)isoxazole (6)

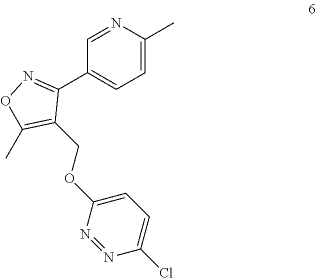 [SMILES: Cc1ccc(-c2noc(C)c2COc2ccc(Cl)nn2)cn1]

with tetrahydropyran-4-amine (7)

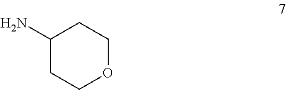

and carbon monoxide in the presence of a base and a palladium catalyst to afford said alogabat (1).

In one embodiment, said palladium catalyst used in the reaction of compound 6 with compound 7 and carbon monoxide is selected from PdCl₂(dppp), PdCl₂(P(p-FC₆H₄)₃)₂, PdCl₂(xantphos), PdCl₂(PPh₃)₂, PdCl₂(dppf), and Pd(amphos)Cl₂.

In a preferred embodiment, said palladium catalyst used in the reaction of compound 6 with compound 7 and carbon monoxide is PdCl₂(dppp).

In one embodiment, said base used in the reaction of compound 6 with compound 7 and carbon monoxide is selected from triethylamine and N-ethyldiisopropylamine.

In a preferred embodiment, said base used in the reaction of compound 6 with compound 7 and carbon monoxide is triethylamine.

In one embodiment, the reaction of compound 6 with compound 7 and carbon monoxide is conducted in a solvent selected from THE, 2-propanol, ethyl acetate, toluene, and ethanol.

In a preferred embodiment, said solvent is selected from THF and 2-propanol.

In a particularly preferred embodiment, said solvent is THF.

In one embodiment, the process according to the invention is as depicted in Scheme 2.

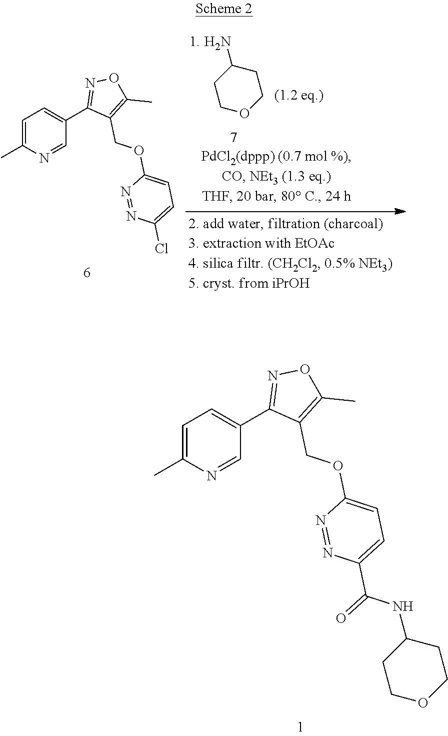 [SMILES: Cc1ccc(-c2noc(C)c2COc2ccc(Cl)nn2)cn1 ; NC1CCOCC1 ; Cc1ccc(-c2noc(C)c2COc2ccc(C(=O)NC3CCOCC3)nn2)cn1]

### EXAMPLES

The invention will be more fully understood by reference to the following examples. The claims should not, however, be construed as limited to the scope of the examples.

The following abbreviations are used in the present text:
NaO^{t}Bu=sodium tert-butoxide; PdCl₂(dppp)=(1,3-Bis(diphenylphosphino)propane)palladium(II) chloride (CAS 59831-02-6); DIPEA=N-ethyldiisopropylamine; THF=tetrahydrofurane; Ti=internal temperature.

### Example 1

### Preparation of 6-chloro-N-tetrahydropyran-4-yl-pyridazine-3-carboxamide (3)

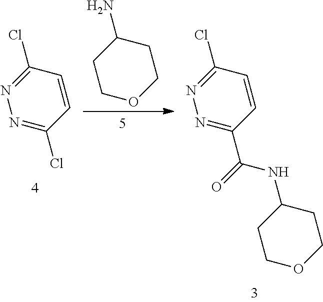

3,6-dichloropyridazine (4) (164.87 g, 1.11 mol) was dissolved in 2-propanol (442 ml) at Ti 40° C. N-ethyldiisopropylamine (150.18 g, 1.16 mol) was added to the yellow solution, followed by tetrahydro-2H-pyran-4-amine (5) (55.97 g, 0.553 ml) and PdCl₂(dppp) (1.08 g, 0.002 mol). The reaction mixture was stirred for about 20 hours under CO pressure (10 bar rel) at 30° C. The resulting blue suspension was filtered and dried to afford the title compound 3 as a white solid (124.3 g). This white solid was dissolved in a mixture of 2-propanol/water (95/5, 250 ml) at 80° C., then cooled within 2 hours to 15° C. and stirred over night. After filtration and drying, the title compound 3 was obtained as a white solid (96.6 g, 71% yield).

### Example 2

### Preparation of 6-[[5-methyl-3-(6-methyl-3-pyridyl)isoxazol-4-yl]methoxy]-N-tetrahydropyran-4-yl-pyridazine-3-carboxamide (Alogabat, 1)

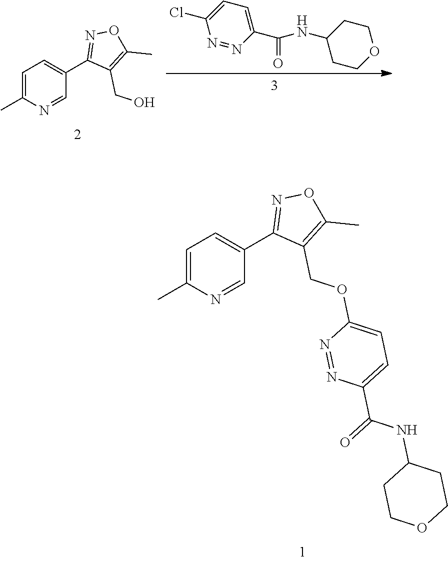

[5-methyl-3-(6-methyl-3-pyridyl)isoxazol-4-yl]methanol 2 (20.0 g, 0.098 mol) was warmed up in 2-methyl-THF (250 ml) to 30° C. To the orange suspension, a suspension of sodium hydride (3.92 g as 60% in oil) in 2-methyl-THF (70 ml) was added. The mixture was stirred 1 hour at 30° C. Then 6-chloro-N-tetrahydropyran-4-yl-pyridazine-3-carboxamide 3 (22.96 g, 0.095 mol) was added in 5 portions over 1 hour. The reaction mixture was stirred over night before it was quenched with water (200 ml). The resulting emulsion was warmed to 50° C. to allow separation of phases. The organic phase was washed with water (30 ml), then filtered over activated charcoal and concentrated. After a solvent exchange with 1-propanol, the resulting suspension was heated to 80° C. to afford a solution, which was cooled to 64° C., seeded and further cooled to 10° C. within 10 hours. After filtration and drying, the title compound 1 was obtained as a white solid (32.0 g, 79.5% yield).

### Example 3

### Preparation of 6-[[5-methyl-3-(6-methyl-3-pyridyl)isoxazol-4-yl]methoxy]-N-tetrahydropyran-4-yl-pyridazine-3-carboxamide (Alogabat, 1)

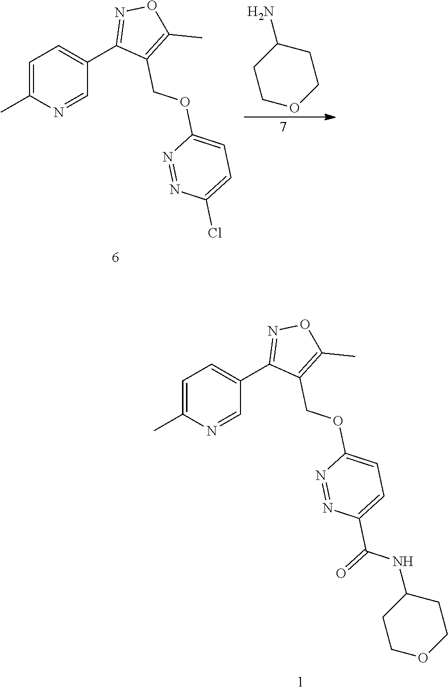 [SMILES: Cc1ccc(-c2noc(C)c2COc2ccc(Cl)nn2)cn1 ; NC1CCOCC1 ; Cc1ccc(-c2noc(C)c2COc2ccc(C(=O)NC3CCOCC3)nn2)cn1]

4-(((6-chloropyridazin-3-yl)oxy)methyl)-5-methyl-3-(6-methylpyridin-3-yl) isoxazole 6 (10 g, 31.1 mmol) was dissolved in THF (70.1 g solution total). The solution was added to an autoclave and the flask was washed with 10 mL of THF. PdCl₂(dppp) (123.3 mg, 209.1 μmol) was added and washed with 2 mL THF. Then triethylamine (4.116 g, 40.47 mmol) and tetrahydro-2H-pyran-4-amine 7 (3.778 g, 37.36 mmol) was added. The resulting brown suspension was stirred for about 23 hours under CO pressure (20 bar rel) at 80° C. then cooled to room temperature. Three batches of the reaction above were combined and water (525 mL) was added. The solution was filtered on charcoal.

Ethyl acetate (880 mL) was added and the phases were separated. The organic layer was washed with 10% sodium chloride aqueous solution (440 mL) then with water (440 mL). The aqueous layer were combined and extracted with ethyl acetate (880 mL). The organic layers were combined and concentrated. The solid was dissolved in dichloromethane containing 0.5% of triethylamine (330 mL). The solution was eluted on silica. The filtrate was evaporated to dryness. The resulting solid was dissolved in 2-propanol (385 mL) at 60° C., seeded and further cooled to 25° C. then stirred for 60 h. After filtration and drying the title compound 1 was obtained as a white solid (28.2 g, 72.2% yield).

1. A process for manufacturing alogabat (1), or a pharmaceutically acceptable salt thereof, 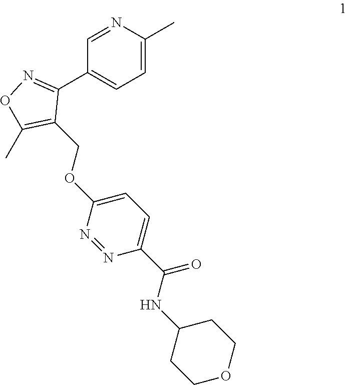 [SMILES: Cc1ccc(-c2noc(C)c2COc2ccc(C(=O)NC3CCOCC3)nn2)cn1]
comprising reacting [5-methyl-3-(6-methyl-3-pyridyl)isoxazol-4-yl]methanol (2) 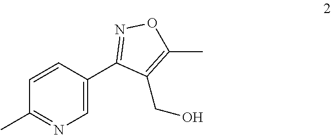
with 6-chloro-N-tetrahydropyran-4-yl-pyridazine-3-carboxamide (3) 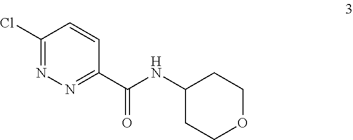 [SMILES: O=C(NC1CCOCC1)c1ccc(Cl)nn1]
in the presence of a base, to afford said alogabat (1).

2. The process according to claim 1, wherein said base is selected from Cs₂CO₃, NaO^{t}Bu, and sodium hydride.

3. The process according to claim 2, wherein said base is sodium hydride.

4. The process according to any one of claims 1 to 3, wherein the process is conducted in an aprotic organic solvent.

5. The process according to claim 4, wherein said aprotic organic solvent is selected from N,N-dimethylacetamide, THF, and 2-methyltetrahydrofuran.

6. The process according to claim 5, wherein said aprotic organic solvent is 2-methyltetrahydrofuran.

7. The process according to any one of claims 1 to 6, wherein alogabat (1) is purified by filtering the reaction mixture over activated charcoal.

8. The process according to any one of claims 1 to 7, wherein alogabat (1) is purified by crystallization.

9. The process according to claim 8, wherein alogabat (1) is crystallized from 1-propanol.

10. The process according to any one of claims 1 to 9, wherein said 6-chloro-N-tetrahydropyran-4-yl-pyridazine-3-carboxamide (3) is obtained by reacting 3,6-dichloropyridazine (4) 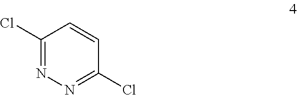
with tetrahydro-2H-pyran-4-amine (5) 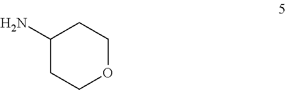
and carbon monoxide in the presence of a base and a palladium catalyst.

11. The process according to any one of claims 1 to 10, which is

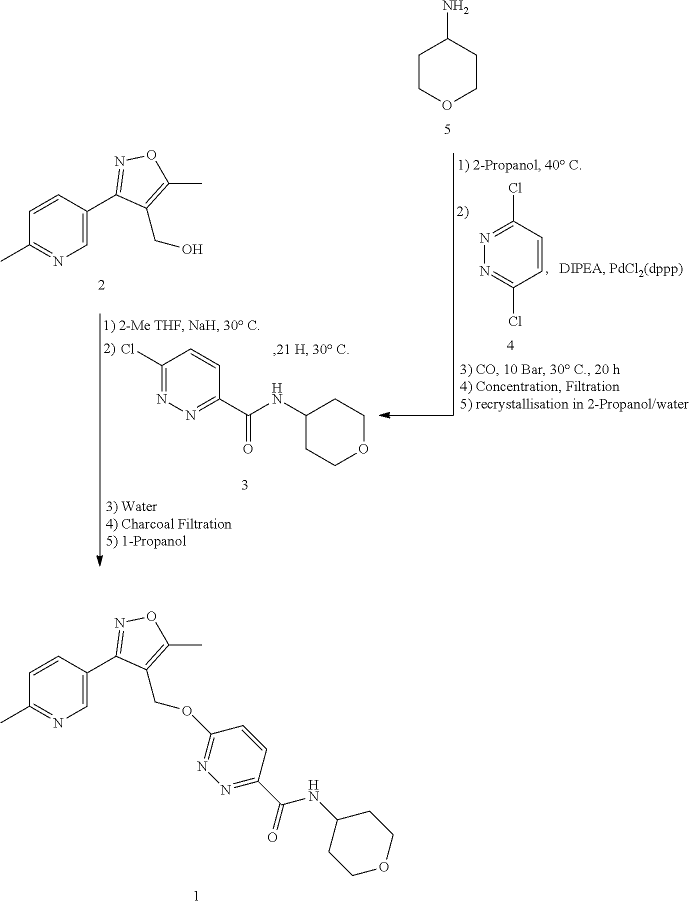

12. A process for manufacturing 6-chloro-N-tetrahydropyran-4-yl-pyridazine-3-carboxamide (3), 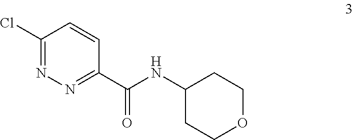 [SMILES: O=C(NC1CCOCC1)c1ccc(Cl)nn1]
comprising reacting 3,6-dichloropyridazine (4) 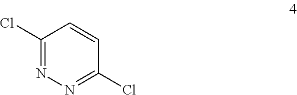
with tetrahydro-2H-pyran-4-amine (5) 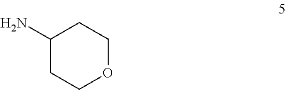
and carbon monoxide in the presence of a base and a palladium catalyst to afford said 6-chloro-N-tetrahydropyran-4-yl-pyridazine-3-carboxamide (3).

13. Use of the process according to claim 12 in the manufacture of alogabat (1).

14. A process for manufacturing alogabat (1), or a pharmaceutically acceptable salt thereof, 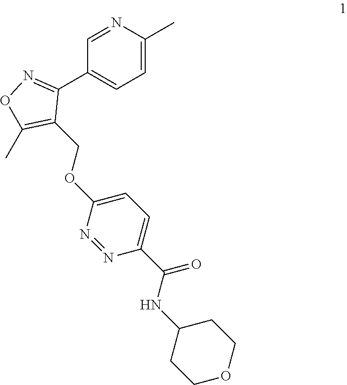 [SMILES: Cc1ccc(-c2noc(C)c2COc2ccc(C(=O)NC3CCOCC3)nn2)cn1]
comprising reacting 4-[(6-chloropyridazin-3-yl)oxymethyl]-5-methyl-3-(6-methyl-3-pyridyl)isoxazole (6) 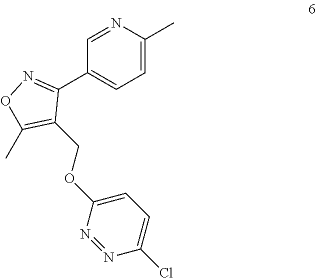 [SMILES: Cc1ccc(-c2noc(C)c2COc2ccc(Cl)nn2)cn1]
with tetrahydropyran-4-amine (7) 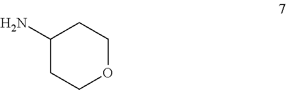
and carbon monoxide in the presence of a base and a palladium catalyst to afford said alogabat (1).

15. The process according to any one of claims 10, 12, and 14, wherein said palladium catalyst is selected from PdCl₂(dppp), PdCl₂(P(p-FC₆H₄)₃)₂, PdCl₂(xantphos), PdCl₂(PPh₃)₂, PdCl₂(dppf), and Pd(amphos)Cl₂.

16. The process according to claim 15, wherein said palladium catalyst is PdCl₂(dppp).

17. The process according to any one of claims 10, 12, and 14 to 16, wherein said base is selected from triethylamine and N-ethyldiisopropylamine.

18. The process according to any one of claims 10, 12, and 14 to 17, wherein said process is conducted in a solvent selected from THF, 2-propanol, ethyl acetate, toluene, and ethanol.

19. The process according to any one of claims 10, 12, and 14 to 17, wherein said process is conducted in a solvent selected from THF and 2-propanol.

20. The process according to claim 14, which is

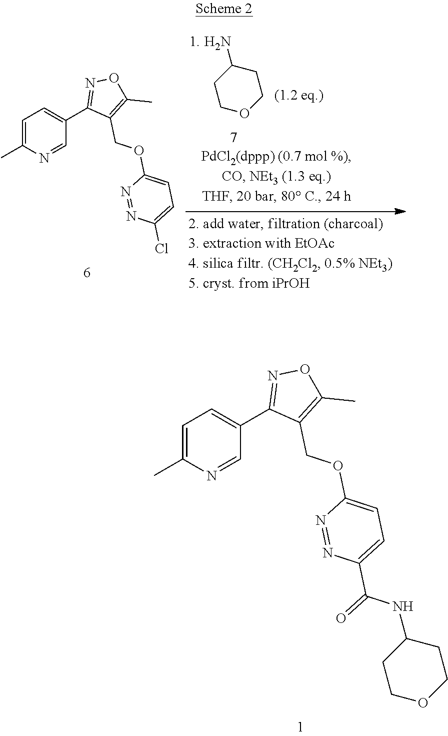 [SMILES: Cc1ccc(-c2noc(C)c2COc2ccc(Cl)nn2)cn1 ; NC1CCOCC1 ; Cc1ccc(-c2noc(C)c2COc2ccc(C(=O)NC3CCOCC3)nn2)cn1]

21. The invention as described hereinbefore.
